(19) United States
(12) Patent Application Publication
(10) Pub. No.: US 2021/0052538 A1
(43) Pub. Date: Feb. 25, 2021

(54) TINTED COLD SORE FORMULATION

(71) Applicant: Carma Laboratories, Inc., Franklin, WI (US)

(72) Inventors: Leslie Curtis, Franklin, WI (US); Keith Edgett, Hartland, WI (US)

(21) Appl. No.: 16/550,000

(22) Filed: Aug. 23, 2019

(51) Int. Cl. A61K 31/245; A61K 9/00; A61K 47/10; A61Q 19/00

(52) U.S. Cl. CPC A61K 31/245; A61Q 19/001; A61K 47/10; A61K 9/0014

(57) ABSTRACT

Provided are skin care compositions that include both therapeutic and cosmetic benefit when applied to the skin and method of use thereof. In particular, provided is an anhydrous composition for treating cold sores, which simultaneously improves the appearance of the cold sore affected skin, therefore avoiding the need for a separate cosmetic treatment.

### BACKGROUND

Skin care compositions, such as cold sore treatments, are commercially available. Conventional cold sore treatments aim to relieve the pain and itching associated with cold sores. Not only are cold sores painful and itchy, the virus causes visible red lesions. The visible symptoms of a cold sore are embarrassing as evidenced by the numerous remedies that cold sore sufferers have devised to disguise the appearance of their sores. Conventional cold sore treatments may be in hydrous or anhydrous forms. Anhydrous compositions may be more advantageous because they adhere to the skin more readily to deliver the active agent to the tissue and are more resistant to removal by water or saliva. However, no hydrous or anhydrous formulations are known to date that may be successfully used to provide both therapeutic (e.g., cold sore treatment) and cosmetic (e.g., disguising the red skin caused by cold sore) effects. In particular, current commercial anhydrous cold sore treatment products do not contain cosmetic ingredients (such as colorants) that hide the red skin lesions without interfering with the medicinal efficacy in treating cold sore. Thus, there is a need for an anhydrous cold sore treatment composition that both treats and conceals the cold sore, especially one that may be stably applied to the skin.

### SUMMARY

In an aspect, the present disclosure provides a skin care composition comprising:

(a) a colorant in an amount of about 0.01% to about 15.00% of the skin care composition by weight;

(b) an active agent in an amount of about 5.00% to about 20.00% of the skin care composition by weight;

(c) a film former in an amount of about 1.00% to about 30.00% of the skin care composition by weight; and

(d) an ointment base in an amount of about 15.00% to about 55.00% of the skin care composition by weight.

In another aspect, provided is a method of treating a skin condition (such as cold sore) comprising applying an effective amount of a skin care composition described herein to a skin of a subject in need thereof.

### DETAILED DESCRIPTION

The present disclosure provides a skin care composition, in particular a composition for treating a cold sore. The composition may be applied to the skin of a subject to both treat the symptoms of the cold sore and simultaneously disguise the undesirable skin appearance (such as red lesions) caused by the cold sore. The composition may be an anhydrous composition. Advantageously, the compositions disclosed herein may be stably applied to the skin to not only provide extended pain and itch relief but also mask the redness associated with cold sores, thus satisfying the therapeutic as well as cosmetic needs of the user in a single formulation.

Before any embodiments of the invention are explained in detail, it is to be understood that the invention is not limited in its application to the details of construction and the arrangement of components set forth in the following description. The invention is capable of other embodiments and of being practiced or of being carried out in various ways.

### 1. Definitions

Unless otherwise defined, all technical and scientific terms used herein have the same meaning as commonly understood by one of ordinary skill in the art. In case of conflict, the present document, including definitions, will control. Suitable methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “comprising,” “include(s),” “including,” “having,” “has,” “contain(s),” “containing,” and variants thereof, as used herein, are open-ended transitional phrases, terms, or words that are meant to encompass the items listed thereafter and equivalents thereof as well as additional items. The singular forms “a”, “and”, and “the” include plural references unless the context clearly dictates otherwise. Where the term “comprising” is used, the present disclosure also contemplates other embodiments “comprising,” “consisting of,” and “consisting essentially of,” the embodiments or elements presented herein, whether explicitly set forth or not. Any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “effective amount,” as used herein, refers to a dosage of an active agent compounds or a composition effective for eliciting a desired effect. This term as used herein may also refer to an amount effective at bringing about a desired in vivo effect in an animal, preferably, a human, such as treatment of a disease, such as a skin disease or condition.

The term “essentially free of” means that a composition contains a component in an amount of less than 1% by weight of the composition. This includes less than 0.5% by weight, less than 0.1% by weight, less than 0.05% by weight, or even less than 0.01% by weight. Compositions “essentially free of” a component also include a composition that is completely free of that component.

The term “skin care composition” as used herein refers to compositions that provide a health benefit to a subject through application to the skin of the subject. For example, the compositions may provide treatment of a skin disease or condition. The skin care compositions may also include those capable of delivering a therapeutic agent useful for treating a condition related or unrelated to the skin of the subject.

The term “topical administration” means administrating an active agent onto the skin of a subject in the need thereof, or delivering an active agent through the skin of a subject in need thereof.

The term “treatment” as used herein in the context of treating a condition (such as a skin disease or condition), pertains generally to treatment and therapy, whether of a human or an animal (e.g. in veterinary applications), in which a desired therapeutic effect is achieved. For example, treatment includes prophylaxis and can ameliorate or remedy the condition, disease, or symptom, or treatment can inhibit the progress of the condition or disease (e.g., reduce the rate of disease/symptom progression or halt the rate of disease/symptom progression).

### 2. Composition

In one aspect, the invention provides a skin care composition comprising:

(a) a colorant in an amount of about 0.01% to about 15.00% of the skin care composition by weight;

(b) an active agent in an amount of about 5.00% to about 20.00% of the skin care composition by weight;

(c) a film former in an amount of about 1.00% to about 30.00% of the skin care composition by weight; and

(d) an ointment base in an amount of about 15.00% to about 55.00% of the skin care composition by weight.

Colorant

The colorant may include one or more coloring materials selected from titanium dioxide, iron oxide, a clay mineral, carmine, a cosmetic dye, and a pearlizing agent. Examples of titanium dioxide colorant include mixtures of titanium dioxide (TiO₂) and isopropyl titanium triisostearate, such as those commercially available under the tradename of BTD-401 (Kobo Products, Inc., NJ; white powder). Examples of the iron oxide colorant include mixtures of iron oxides and isopropyl titanium triisostearate, such as those commercially available under the tradename of BYO-12 (Kobo Products, Inc., South Plainfield, N.J.; e.g., CI77491 (red powder), CI77492 (yellow powder), or CI77499 (black powder)). A pearlizing agent may reflect light waves and may provide an opaque pearl-like aesthetic appearance. Examples of pearlizing agents include various pearlescent pigments and colorants known in the art for their use in cosmetics (such as titanium dioxide-based or mica-based powders, and mixtures thereof). Examples of commercial pearlizing agent include KTZ®Gold Lame-I2 (Kobo Products, Inc., NJ; golden powder, mixture of mica, titanium dioxide, iron oxides, and isopropyl titanium triisostearate). Examples of cosmetic dyes include those approved by U.S. Food and Drug Administration (FDA) for use in cosmetics and food, including various drug and cosmetic (D&C) organic dyes and pigments known in the art (such as FD&C Yellow #5, D&C Red #33).

In some embodiments, the colorant may include titanium dioxide, iron oxide, a pearlizing agent, or a combination thereof. In particular embodiments, the colorant includes BTD-401, one or more BYO-12 materials (read, yellow, and black), KTZ®Gold Lame-I2, and combinations thereof.

The skin care compositions disclosed herein may comprise about 0.01% to about 20.00% by weight of a colorant. The compositions may comprise at least 0.10%, at least 0.50%, at least 1.00%, at least 2.00%, at least 3.00%, at least 4.00%, at least 5.00%, at least 6.00%, at least 7.00%, at least 8.00%, at least 9.00%, at least 10.00%, at least 11.00%, at least 12.00%, at least 13.00%, at least 15.00%, at least 17.00%, or at least 19.00% by weight of a colorant. The compositions may comprise less than 20.00%, less than 18.00%, less than 16.00%, less than 14.00%, less than 13.00%, less than 12.00%, less than 11.00%, less than 10.00%, less than 9.00%, less than 8.00%, less than 7.00%, less than 6.00%, less than 5.00%, less than 4.00%, less than 3.00%, less than 2.00%, or less than 1.00% by weight of a colorant. The compositions may comprise about 0.01% to about 15.00%, 0.50% to about 14.00%, about 1.00% to about 14.00%, about 1.00% to about 13.00%, about 1.00% to about 12.00%, about 1.00% to about 11.00%, about 1.00% to about 10.00%, about 1.00% to about 9.00%, about 1.00% to about 8.00%, about 1.00% to about 7.00%, about 1.00% to about 6.00%, or about 1.00% to about 5.00% by weight of a colorant. In some embodiments, the compositions comprise from about 1.00% to about 10.00% or about 1.00% to about 5.00% by weight of a colorant.

In some embodiments, the compositions include BTD-401 in an amount of about 1.00% to about 6.00% by weight, such as about 1.25%, about 1.50%, about 2.00%, about 2.50%, about 3.00%, about 3.50%, about 4.00%, about 4.50%, about 5.00%, or about 5.25% by weight. In some embodiments, the compositions include one or more BYO-12 materials (read, yellow, and black), each in an amount of about 0.01% to about 3.00% by weight, such as about 0.01%, about 0.05%, about 0.08%, about 0.15%, about 0.50%, about 0.80%, about 1.00%, about 1.50%, or about 2.00% by weight. In some embodiments, the compositions include KTZ®Gold Lame-I2 in an amount of about 0.01% to about 3.00% by weight, such as about 0.25%, about 0.50%, about 1.00%, about 1.50%, about 2.00%, or about 2.50% by weight.

Active Agent

The active agent may be a therapeutic agent that can be administered through application to the skin, and may include one or more known pharmaceutically active compounds. For example, the active agent may be an analgesic agent, an anti-microbial agent, an anti-viral agent, an immunosuppressive agent, a corticosteroid, a non-steroidal anti-inflammatory drug (NSAID), a hair-growth agent, an ophthalmic agent, a dermatological agent, an antifungal agent, an anti-bacterial agent, a wound-healing agent, and a combination thereof.

Examples of anti-microbial agents include erythromycin, clindamycin, bacitracin, tobramycin, neomycin, neosporin, polymycin B, benzoyl peroxide, mupirocin, cephalosporins, and penicillins, gentamicin, tetracyclines, streptomycin, kanamycin, vanomycin, rifampin, minocycline, doxycycline, lincomycin, temflaxocine, ciproflaxocine, levoflaxocine, gatiflaxocine, quinolones, fluoroquinolones, sulfonamides, and aminoglycosides.

Examples of anti-viral agents include abacavir, acyclovir, arbidine, cidofavir, doluregravir, edoxudine, famciclovir, ganciclovir, oseltamivir, valaciclovir. Examples of immunosuppressive agents include methatrexate, cyclosporins, tacrolimus, pimecrolimus, sirolimus, and everolimus. Examples of corticosteroids (as anti-inflammatory or anti-pruitic agents) include desonide, hydrocortisone, cortisone, clocortolone, clobetasol, desoximetasone, dexamethasone, fluocinolone, fluocinonide, prednisolone, diflorasone, betamethasone, triamcinolone, and derivatives thereof. Examples of non-steroid anti-inflammatory drugs include ibuprofen, diclofenac, naproxen, melcoxicam, piroxicam, and methyl salicylate.

Examples of hair growth agents include minoxidil, finasteride, and spironolactone. Examples of ophthalmic agent include moxifloxacin, atropine, travoprost, olopatadine, natamycin, nepafenac, tropicamide, apraclonidine, bimatoprost, ketorolac, nedocromil, levobunolo, epinastine, and ofloxacine. Examples of dermatological agents include 5-fluorouracil, azelaic acid, ivermectin, bromonidine, calcipotriene, retapamulin, salicylic acid, and ingenol mebutate.

Examples of anti-fungal agents include bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, terbinafine, ciclopirox, flucytosine, griseofulvin, tolnaftate, amphotericin B, candicidin, filipin, hamycin, natamycin, nystatin, rimocidin, anidulafungin, caspofungin, micafungin, and crystal violet.

Examples of active agents promoting wound healing include epidermal growth factor, transforming growth factor-α, transforming growth factor-β, heptocyte growth factor, vascular endothelial growth factor, platelet derived growth factor, fibroblast growth factor, and keratinocyte growth factor.

In some embodiments, the active agent is an analgesic agent. Examples of analgesic agents include, but are not limited to, benzocaine, lidocaine, tetracaine, dibucaine, prilocaine, capsaicin, fentanyl, pregabalin, gabapentin, carbamazepine, oxcarbazepine, duloxetine, venlafaxine, bupropion, an opioid, and a combination thereof. In particular embodiments, the active agent is benzocaine.

The skin care compositions disclosed herein may comprise about 1.00% to about 30.00% by weight of an active agent. The compositions may comprise at least 1.00%, at least 2.00%, at least 4.00%, at least 6.00%, at least 8.00%, at least 10.00%, at least 12.00%, at least 14.00%, at least 16.0%, at least 18.0%, at least 20.0%, at least 22.00%, at least 24.00%, at least 26.00%, or at least 28.00% by weight of an active agent. The compositions may comprise less than 30.00%, less than 28.00%, less than 26.00%, less than 24.00%, less than 22.00%, less than 20.00%, less than 18.00%, less than 16.00%, less than 14.00%, less than 12.00%, less than 10.00%, less than 8.00%, less than 6.00%, less than 4.00%, or less than 2.00% by weight of an active agent. The compositions may comprise about 1.00% to about 25.00%, about 5.00% to about 25.00%, about 5.00% to about 20.00%, about 5.00% to about 15.00%, or about 5.00% to about 10.00% by weight of an active agent. In some embodiments, the compositions comprise from about 5.00% to about 20.00% or about 5.00% to about 10.00% by weight of an active agent.

In some embodiment, the active agent is benzocaine. The skin care compositions disclosed herein may comprise about 1.00% to about 25.00% by weight of benzocaine. The compositions may comprise at least 1.00%, at least 2.00%, at least 3.00%, at least 4.00%, at least 5.00%, at least 6.00%, at least 7.00%, at least 8.00%, at least 9.00%, at least 10.00%, at least 11.00%, at least 12.00%, at least 13.00%, at least 14.00%, at least 15.00%, at least 16.00%, at least 17.00%, at least 18.00%, at least 19.00%, at least 20.00%, at least 21.00%, at least 21.00%, at least 22.00%, at least 23.00%, or at least 24.00% by weight of benzocaine. The compositions may comprise less than 25.00%, less than 24.00%, less than 23.00%, less than 22.00%, less than 21.00%, less than 20.00%, less than 19.00%, less than 18.00%, less than 17.00%, less than 16.00%, less than 15.00%, less than 14.00%, less than 13.00%, less than 12.00%, less than 11.00%, less than 10.00%, less than 9.00%, less than 8.00%, less than 7.00%, less than 6.00%, less than 5.00%, less than 4.00%, less than 3.00%, or less than 2.00% by weight of benzocaine. The compositions may comprise about 5.00% to about 24.00%, about 5.00% to about 22.00%, about 5.00% to about 20.00%, about 7.00% to about 20.00%, about 7.00% to about 18.00%, about 7.00% to about 16.00%, about 7.00% to about 12.00%, or about 7.0% to about 10.00% by weight of a benzocaine. In some embodiments, the compositions comprise from about 7.00% to about 20.00% by weight of benzocaine. In some embodiments, the compositions comprise about 7.0%, about 8.0%, about 9.0%, about 10.0%, about 11.0%, about 12.0%, about 13.0%, about 14.0%, or about 15.0% by weight of benzocaine. In some embodiments, the compositions comprise about 10.0% by weight of benzocaine.

Film Former

The film former may facilitate the skin care composition to form a thin film on the skin, and may provide stable adherence of the active agent and the colorant to the skin. Remarkably, the film formers of the compositions disclosed herein may maintain a thin film of a disclosed composition on a skin having cold sore symptoms, therefore maintaining close contact between the composition (including the colorant and the active agent) and the skin for an extended period, which may result in improved treatment of the cold sore and simultaneously provide a cosmetic effect in hiding the undesirable skin appearances (e.g., unpleasant redness) caused by cold sore. Advantageously, the combination of the active agent (such as an analgesic agent, or benzocaine in particular), the colorant, and the film former disclosed herein may allow for an even distribution of the composition on the skin, thereby providing an uniform therapeutic as well as cosmetic treatment of the skin. In particular embodiments, the film formers in combination with the other ingredients of the composition disclosed herein may provide extended pain and itch relief and mask the redness associated with cold sores.

The film former may include a polymeric material, an organic material, or combinations thereof. Examples of polymeric film former include 4,4′-dii socyanatodicyclohexylmethane polypropylene glycol polymer (CAS registry No. 9042-82-4, also named poly(oxy(methyl-1,2-ethanediyl)), alpha-hydro-omega-hydroxy-, polymer with 1,1′-methylenebis(4-isocyanatocyclohexane)), which may be commercially available under the name of polyolprepolymer-14 or PPG-51 SMDI copolymer (e.g., Polyolprepolymer-14, supplied by Barnet Products Corp). Suitable organic materials include esters having one or more long aliphatic chains, such as esters formed by C16-C22 fatty alcohol and/or C16-C22 fatty acids. Examples of organic materials include behenyl olivate, behenyl erucate, dibehenyl fumarate, bis-behenyl/isostearyl/phyosteryl dimer dilinoleyl dimer dilinoleate, phytosteryl/behenyl/octyldodecyl lauroyl glutamate, bis-behenyl/phytosteryl dimer dilinoleate, and a combination thereof. Suitable film former may be a mixture of one of more polymeric materials and one or more organic materials as disclosed herein. In some embodiments, the film former may include polyolprepolymer-14, behenyl olivate, or a mixture thereof.

The skin care compositions disclosed herein may comprise about 0.50% to about 40.00% by weight of a film former. The compositions may comprise at least 1.00%, at least 5.00%, at least 10.00%, at least 15.00%, at least 20.00%, at least 25.00%, at least 30.00%, or at least 35.00% by weight of a film former. The compositions may comprise less than 40.00%, less than 35.00%, less than 30.00%, less than 25.00%, less than 20.00%, less than 15.00%, or less than 10.00% by weight of a film former. The compositions may comprise about 1.00% to about 40.00%, about 1.00% to about 30.00%, about 5.00% to about 30.00%, about 5.00% to about 25.00%, about 5.00% to about 20.00%, or about 5.00% to about 10.00% by weight of a film former. In some embodiments, the compositions comprise from about 5.00% to about 25.00% or about 5.00% to about 10.00% by weight of a film former. In some embodiments, the compositions comprise about 5.00%, about 8.00%, about 10.00%, about 15.00%, about 18.00%, about 20.00%, or about 25.00% by weight of a film former. In particular embodiments, the compositions comprise about 8.00% by weight of a film former.

In some embodiments, the compositions include polyolprepolymer-14 and behenyl olivate, each independently in an amount of about 1.00% to about 10.00% of the skin care composition by weight. For example, each of the polyolprepolymer-14 and behenyl olivate may independently be present at about 1.00%, about 2.00%, about 3.00%, about 4.00%, about 5.00%, about 6.00%, about 7.00%, about 8.00%, about 9.00%, or about 10.00% by weight. In some embodiments, the polyolprepolymer-14 and behenyl olivate may both be present at about 2.00%, about 3.00%, about 4.00%, about 5.00%, about 6.00%, about 7.00%, about 8.00% by weight. In some embodiments, the polyolprepolymer-14 and behenyl olivate may both be present at about 4.00% by weight of the skin care composition.

Ointment Base

Ointment bases may give a soft semisolid form to the skin composition disclosed herein, which then may be applied to the surface of the skin. Suitable ointment bases include known materials used in personal care products and drug delivery, and may provide beneficial characteristics (such as emollience and occludedness). Suitable ointment bases may be inert, stable, nonirritating and nonsensitizing materials. Suitable ointment bases may include oleaginous ointment bases, such as white ointment, yellow ointment, cetyl esters wax, paraffins, petrolatum, white petrolatum, white wax, yellow wax, beeswax, and the like and mixtures thereof. In some embodiments, the ointment bases include petrolatum (e.g., CAS Registry No. 8009-03-8).

The skin care compositions disclosed herein may comprise about 10.00% to about 60.00% by weight of an ointment base. The compositions may comprise at least 10.00%, at least 15.00%, at least 20.00%, at least 25.00%, at least 30.00%, at least 35.00%, at least 40.00%, at least 45.00%, at least 50.00%, or at least 55.00% by weight of an ointment base. The compositions may comprise less than 60.00%, less than 55.00%, less than 40.00%, less than 35.00%, less than 30.00%, less than 25.00%, or less than 20.00% by weight of an ointment base. The compositions may comprise about 10.00% to about 55.00%, about 15.00% to about 55.00%, about 15.00% to about 50.00%, about 20.00% to about 50.00%, about 30.00% to about 50.00%, or about 40.00% to about 50.00% by weight of an ointment base. In some embodiments, the compositions comprise from about 15.00% to about 55.00% or about 15.00% to about 45.00% by weight of a film former. In some embodiments, the compositions comprise about 40.00%, about 45.00%, or about 50.00% by weight of an ointment base. In some embodiments, the compositions comprise about 45.00% by weight of a film former. In particular embodiments, the ointment base is petrolatum, which is present in the skin care composition at an amount of about 40.00% to about 50.00% by weight.

Anhydrous Composition

The skin care composition may be essentially free of water. For example, the skin care composition may contain less than 1.00%, less than 0.10%, or less than 0.01% by weight of water. In some embodiments, the skin care composition as disclosed herein is an anhydrous composition, which is free of any detectable amount of water. Advantageously, the present skin care composition may achieve equal or higher efficacy in treating a cold sore as compared to known anhydrous products (e.g., Carmex® Cold Sore Treatment External Analgesic, Carma Laboratories, Inc., Franklin, Wis.), and simultaneously provide cosmetic benefit that eliminate the need of the user to separately disguise the undesirable skin appearance caused by a cold sore.

In a particular embodiments, provided is a skin care composition, comprising

(a) a colorant in an amount of about 1.00% to about 10.00% of the skin care composition by weight;

(b) benzocaine in an amount of about 7.00% to about 20.00% of the skin care composition by weight;

(c) polyolprepolymer-14 and behenyl olivate, each independently in an amount of about 1.00% to about 10.00% of the skin care composition by weight; and
(d) petrolatum in an amount of about 40.00% to about 50.00% of the skin care composition by weight.

Additional Ingredients

The skin care compositions disclosed here may further comprise one or more additional components. In some embodiments, the compositions may further comprise an additive selected from a surfactant, an oil absorber, a slip modifier, a flavorant, and a combination thereof.

The surfactant may provide surface wetting and/or facilitate the solubilization of the other ingredients of the composition, such as the active agent. Suitable surfactants include various known nonionic surfactants used in skin care products (such as hand and body creams). In general, the suitable surfactants do not cause skin damage or irritation during extended application to the skin. For example, the nonionic surfactant may be selected from the group consisting of ethylene oxide adducts of C8 to C22, preferably C8 to C16, more preferably C8 to C12 alcohols, ethylene oxide/propylene oxide adducts of ethylene glycol, alkylene glycols or mixtures thereof. In some embodiments, the surfactant may include a polyethylene glycol (PEG) with an average molecular weight of about 300 g/mol (PEG-300) to about 10,000 g/mol (PEG-10,000). In some embodiments, the surfactant is selected from PEG-300, PEG-600, PEG-800, PEG-1000, PEG-2000, PEG-5000, PEG-10000, and a combination thereof. In some embodiments, the polyethylene glycol is PEG-600, such as Carbowax™ Sentry™ Polyethylene Glycol 600 NF (CAS Registry No. 25322-68-3, Dow Chemical Co.).

The compositions disclosed herein may comprise about 5.00% to about 25.00% by weight of a surfactant. The compositions may comprise at least 5.00%, at least 10.00%, at least 15.00%, or at least 20.00% by weight of a surfactant. The compositions may comprise less than 25.00%, less than 20.00%, less than 15.00%, or less than 10.00% by weight of a surfactant. The compositions may comprise about 5.00% to about 20.00%, about 10.00% to about 20.00%, or about 10.00% to about 15.00% by weight of a surfactant. In some embodiments, the compositions comprise from about 10.00%, about 15.00%, or about 20.00% by weight of a surfactant.

In some embodiments, the compositions comprise one or more polyethylene glycols in an amount of about 10.0%, about 12.0%, about 15.0%, about 18.0%, or about 20.0% by weight. In some embodiments, the compositions comprise one or more polyethylene glycols (such as PEG-600) in an amount of about 15.0% by weight.

The oil absorber may provide improved aesthetic appearance, stability, and/or skin feel (such as smoothness and silkiness) of the skin care compositions disclosed herein. The oil absorber may also reduce oil on the skin and reduce irritation, therefore enhancing the therapeutic and cosmetic effects of the present composition. Suitable oil absorbers include, for example, micronized powders known for their use to provide a balanced, natural finish and healthy appearance on skin. The oil absorber may include a wax and calcium silicate. Commercial oil absorbers include, for example, Microsorb 944S (polyethylene/calcium silicate/silica), Microsorb 988S (synthetic wax/calcium silicate/silica), and Naturesorb 1000 (copernicia cerifera (Carnauba) wax/calcium silicate) supplied by Micro Powders, Inc. (Tarrytown, N.Y.). In some embodiments, the oil absorber is Microsorb 988S, which is a white powder (mean particle size 22.0-30.0 microns) containing a blend of synthetic wax (CAS Registry No. 8002-74-2), calcium silicate (CAS Registry No. 1344-95-2), and silica (CAS Registry No. 112945-52-5).

The compositions disclosed herein may comprise about 1.00% to about 20.00% by weight of an oil absorber. The compositions may comprise at least 2.00%, at least 3.00%, at least 4.00%, at least 5.00%, at least 6.00%, at least 7.00%, at least 8.00%, at least 9.00%, at least 10.00%, at least 12.00%, at least 14.00%, at least 16.00%, or at least 18.00% by weight of an oil absorber. The compositions may comprise less than 19.00%, less than 17.00%, less than 15.00%, less than 13.00%, less than 11.00%, less than 9.00%, less than 8.00%, less than 7.00%, less than 6.00%, less than 5.00%, less than 4.00%, less than 3.00%, or less than 2.00% by weight of an oil absorber. The compositions may comprise about 1.00% to about 15.00%, about 1.50% to about 15.00%, about 2.00% to about 15.00%, about 2.00% to about 10.00%, or about 2.00% to about 5.00% by weight of an oil absorber. In some embodiments, the compositions comprise from about 1.50% to about 15.00% by weight of an oil absorber. In some embodiments, the compositions comprise about 2.00%, about 2.50%, about 3.00%, about 3.50%, about 4.00%, about 4.50%, about 5.00%, or about 5.50% by weight of an oil absorber.

In some embodiments, the compositions comprises Microsorb 988S in an amount of about 1.00% to about 15.00% by weight, such as about 3.00%, about 3.50%, about 4.00%, about 4.50%, about 5.00%, or about 5.50% by weight. In particular embodiments, the compositions comprise Microsorb 988S in an amount of about 3.50% by weight.

The slip modifier may offer a ball-bearing effect which may impart finished composition with a silky texture and enhanced slip which promotes better blendability on the skin. The slip modifier also may scatter light to diminish the look of fine lines on the skin, while letting enough light through so the look of the skin is natural. Suitable slip modifiers include, for example, cosmetic powders and polymer microspheres known in the art. Commercial slip modifiers include RonaFlair® Extender W cosmetic pigment (a white to pale yellow powder, EMD Chemicals, Philadelphia, Pa.), which contains mica (mineral, CAS Registry No. 12001-26-2, 54%-59% by weight) and titanium dioxide (CAS Registry No. 13463-67-7, 41%-46% by weight). Other commercial slip modifiers include BPD-500 microspheres (white to pale yellow powder, Kobo Products, Inc., South Plainfield, N.J.), which contains hexamethylene diisocyanate (HDI)/trimethylol hexyllactone crosspolymer (CAS Registry No. 129757-76-2, 97.0%-99.0% by weight) and silica (CAS Registry No. 7631-86-9, 1.0%-3.0% by weight). Other suitable slip modifiers include calcium sodium borosilicate and polymethyl methacrylate. In some embodiments, the skin compositions disclosed herein comprise a mica/titanium dioxide powder (such as RonaFlair® Extender W cosmetic pigment), or polymer microspheres (such as BPD-500), or both as slip modifiers.

The compositions disclosed herein may comprise about 1.00% to about 35.00% by weight of a slip modifier. The compositions may comprise at least 2.00%, at least 5.00%, at least 10.00%, at least 15.00%, at least 20.00%, at least 25.00%, or at least at least 30.00% by weight of a slip modifier. The compositions may comprise less than 30.00%, less than 25.00%, less than 20.00%, less than 15.00%, less than 10.00%, or less than 5.00% by weight of an a slip modifier. The compositions may comprise about 2.00% to about 30.00%, about 5% to about 30.00%, about 5% to about 25.00%, about 5.00% to about 20.00%, or about 5.00% to about 15.00% by weight of a slip modifier. In some embodiments, the compositions comprise from about 5.00% to about 20.00% or about 5.00% to about 15.00% by weight of a slip modifier. In some embodiments, the compositions comprise about 10.00%, about 12.00%, about 14.00%, about 16.00%, about 18.00%, or about 20.00% by weight of a slip modifier.

In some embodiments, the slip modifier is a mixture of a mica/titanium dioxide powder (such as RonaFlair® Extender W cosmetic pigment) and polymer microspheres (such as BPD-500), each in an amount about 1.00% to about 15.00% by weight of the skin care composition. In particular embodiments, the composition comprises a mixture of RonaFlair® Extender W cosmetic pigment and BPD-500, each in an amount of about 1.00% to about 15.00% by weight, such as about 5.00%, about 7.00%, or about 10.00% by weight.

The skin care compositions disclosed herein may also include one or more flavorants, such as the natural or artificial flavor compounds (e.g., those providing fruit or mint flavors) known in the art. The flavorant may improve the sensory quality (e.g., pleasant taste and smell) of the compositions, which is of particular benefit if the composition is used orally. In some embodiments, the compositions comprise a peppermint flavorant, such as FY-996 (Ungerer & Co., Lincoln Park, N.J.). The compositions may comprise about 0.10% to about 10.00% by weight of a flavorant. In some embodiments, the compositions comprise about 0.20%, about 0.30%, about 0.50%, about 1.00%, or about 2.00% by weight of a flavorant.

### 3. Method

In another aspect, the present disclosure provides a method of treating a skin condition, which comprises applying an effective amount of a skin care composition disclosed herein to a skin of a subject in need thereof. The method may result in topical administration of the active agent to the subject.

In some embodiments, the subject is a patient having a skin disease, such as bacterial infection or a viral infection. In some embodiments, the subject has cold sore, such as the infection caused by the herpes simplex virus (HSV-1) near the mouth (e.g., around the border of the lips) or on other areas of the face. The amount of the compositions applied to the skin of the subject may depend on the area of the skin to be treated and the physical conditions of the subject (e.g., age and body weight). The compositions may be applied at a pre-determined frequency (e.g., once daily) depending on the concentration, the skin absorption rate, and the pharmacokinetic properties of the active agent.

In some embodiments, the compositions comprises benzocaine, and may be used to treat a cold sore. For example, such compositions may be applied to the skin of the subject once daily, twice daily, or even more frequently as needed to treat the symptoms of a cold sore. In some embodiments, the compositions may deliver about 0.1 mg/kg to about 1000 mg/kg of benzocaine to the skin, including about 1.0 mg/kg to about 500 mg/kg, about 1.0 mg/kg to about 200 mg/kg, or about 1.0 mg/kg to about 100 mg/kg.

As described herein, the present skin care compositions may provide both therapeutic (topical administration of an active agent) and cosmetic (modifying skin appearance) benefits to a subject in a single application, thus eliminating the need to apply separate treatments to the skin. In particular embodiments, the composition is an anhydrous composition that provides both effective treatment of a cold sore and a satisfactory cosmetic effect to improve the appearance of treated skin.

The following non-limiting examples illustrate the compositions of the present disclosure and method of use thereof.

### EXAMPLES

### Example 1. Preparation of Tinted Cold Sore Formulation

A mixture of PEG-600, benzocaine, and Polyolprepolymer-14 is prepared in a beaker large enough to contain the entire batch and is set aside (Batch Beaker). Titanium Dioxide, Yellow Iron Oxide, Red Iron Oxide, and Black Iron Oxide are weighed out into a beaker. The percentage of each colorant is increased by 10% to account for loss during blending. Pigments are mixed together with a spatula and poured into the blending container of a powder mixer. The pigment mixture is pulsed on low speed until the color is homogenous. The pigments are re-weighed to assure the correct amount is yielded. The contents of the Batch Beaker are mixed for 10-15 minutes to ensure that benzocaine has dissolved and the ingredients are homogenous. The blended pigments are added to the Batch Beaker with mixing until well dispersed. Petrolatum and behenyl olivate are added to the Batch Beaker and heated to 85° C. with mixing. When the batch reaches a temperature of 85° C., Microsorb 988S, BPD-500, RonaFlair® Extender White W cosmetic pigment, and KTZ®Gold Lame-12 are added. The batch is mixed until all ingredients are homogenous, and the temperature is 85° C. The batch is then cooled to 65° C. while still mixing. When 65° C. is reached, peppermint flavorant is added. The batch is mixed until all ingredients are homogenous and is poured off into storage container(s).

The weight percent of each ingredient in the composition is shown in Table 1 below.

TABLE 1

| Ingredient | | Weight % of |
|---|---|---|
| Trade Name | INCI Name | composition |
| PEG-600 | PEG 600 | 15.00% |
| Benzocaine | Benzocaine | 10.00% |
| Polyolprepolymer-14 | PPG-51 SMDI Copolymer | 4.00% |
| BTD-401 | TiO2 and Isopropyl Titanium Triisostearate (ITT) | 3.00% |
| BYO-12 | Yellow Iron Oxide (CI77492) and ITT | 0.80% |
| BBO-12 | Black Iron Oxide and ITT | 0.08% |
| BRO-12 | Iron Oxides (CI77491) and ITT | 0.38% |
| Petrolatum | Petrolatum | 44.69% |
| Behenyl Olivate | Behenyl Olivate | 4.00% |
| Microsorb 988S | Synthetic Wax Calcium Silicate Silica | 3.50% |
| BPD-500 | HDI/Trimethylol Hexyllactone Crosspolymer | 7.00% |
| | (and) Silica | |
| RonaFlair ® Ext. | Mica (and) Titanium Dioxide | 7.00% |
| White | | |
| KTZ ® Gold Lame-12 | Mica (and) Titanium Dioxide (and) Iron Oxides | 0.25% |
| | (and) ITT | |
| Peppermint Flavor | Ungerer Peppermint Flavor FY-9996 | 0.30% |
| | | 100.00% |

### Example 2. Evaluation of Tinted Cold Sore Formulation on Subjects

Study Material

Representative tinted cold sore treatment compositions (“test product”) as described herein are tested, the result of which are reviewed along with other assessments needed for regulatory and safety approval.

Subject Recruitment and Selection

Fifty subjects who are active cold sore sufferers with redness present as part of cold sores located on or around the lips are asked to apply the test product according to the provided use instructions. Subjects include 20%-30% males and 70%-80% females, ages 18 through 65 years old. Individuals are required to complete a preliminary medical history and screening document as mandated. Individuals in generally good health and free of any health problems (including neurological, dermatological, or systemic disorder that would interfere with the purpose, conduct or integrity of the study), as well as individuals with symptoms of a cold sore are included. Participating individuals must not be using cold sore treatments or cosmetics (other than what is provided by the study director) to cover the appearance of cold sores 48 hours prior to, or during the study. Individuals expressing a negative reaction to test materials and females who are pregnant, lactating, have been pregnant, or given birth within the six-month period immediately preceding the study commencement are excluded.

A sufficient number of male and female volunteers between the ages of 18 and 65 years are recruited to assure a minimum of fifty subjects to complete the study. As an example, 10 to 15 male subjects and 35 to 40 female subjects may be recruited.

Procedure

All fifty subjects are examined by a trained technician in order to qualify the appearance of a cold sore. The test products are weighed and recorded prior to study initiation and again upon study completion. The subjects are instructed to use the test product according to instructions as follows.

Using clean, dry hands apply 1 to 2 small dollops of product to the tip of the finger and gently apply the cold sore treatment evenly to the surface of the cold sore. Gently spread the treatment to ensure even coverage over and around the cold sore and inflamed area (areas of redness). Use as much product as is necessary to provide even coverage. Wash hands thoroughly after application.

A sufficient quantity of the product are applied to cover cold sore affected area/redness. The test product is applied under the supervision of trained technicians.

Subjects are asked to remain at the test facility for the duration of the study. Objective expert grading is performed prior to application of the test product, instantly (X sec<60 sec), immediately after application (60 seconds) and again after 30 minutes. Subjects are not allowed to drink, eat food, use lip balm or wipe lips/cold sores areas during the duration of the study.

At each evaluation time point (Baseline, Instant<60 sec, Immediate (60 sec), and 30 minutes), all participants are asked to complete a self-assessment questionnaire. Efficacy is photographically documented on a subset of ten subjects from within the panel at baseline, instantly (X sec<60 sec), immediately after (60 seconds) and again after 30 minutes post application of the test product. At the conclusion of the study, the data are reviewed by a dermatologist to confirm the results and conclusions.

Visual Expert Grading Scale

Expert Visual Grading Scale allows the Trained Clinical Evaluator to grade the subject for number of visual and tactile parameters including redness using the same 11-point intensity grading scale used at baseline where 0=Best Condition Imaginable and 10=Worst Condition Imaginable. The scale includes a color gradient for grading of redness, 0 being closest to flesh tone and 10 being the most red. The parameters include dryness and immediate/instant reduction (hides/masks) redness.

High Resolution Matched Scientific Photography™ & PhotoGrammetrix®

Each stage in the progression of treatment are photographically documented (High Resolution Matched Scientific Photography™) under controlled conditions and evaluated through PhotoGrammetrix® image analysis (AMA Laboratories, Inc., New City, N.Y.). High Resolution Matched Scientific Photography™ are taken at baseline and again after 60 seconds and after 30 minutes after product application. The photographs are taken prior to the subjective evaluation portion of the test.

Detailed, high resolution before and after product application digital photographs are taken, with fixed camera background, distances, angles, settings, lighting, panelist positioning, color bars, white balance, standardized and digitally certified unretouched. Each stage in the progression of treatment are photographically documented as a visual record.

Photographs are evaluated via image analysis software which allows capturing the areas of involvement (cold sore area). The analysis takes into the account the geometry (px²−size of the cold sore, expressed as cold sore related pixels per area of involvement) and color of the cold sore (CIE L*a*b* Color Scale—color fading). An increase in the L* coordinate indicates lightening, an increase in the a* coordinate indicates reddening, and an increase in the b* coordinate indicates yellow. A decrease in the L* coordinate indicates darkening, a decrease in the a* coordinate indicates green, and a decrease in the b* coordinate indicates blue.

Subjective Questionnaire

Subjects are instructed to complete self-assessment questionnaires addressing consumer perception after 60 seconds and after 30 minutes post-application of the test product (including treatment of cold sore on the lip and/or around the mouth). A 5-point scale will be employed for the following questions—the product instantly hides redness associated with the cold sore; the product provided fast relief of the pain and itch associated with the cold sore; upon application, the product masks/hides/conceals redness. The 5-point scale includes the following answer choices: STRONGLY AGREE, AGREE, DO NOT KNOW, DISAGREE, STRONGLY DISAGREE.

Statistical and Photo Analysis

The source data are expert grading values, subjective questionnaire responses as well as PhotoGrammetrix™ analysis results, which are totaled and reported as average scores. Moreover the obtained data are quoted as percent differences from baseline for each of the previously described time points (where applicable). Photographs are placed side by side as to compare the photographs of before and after use of the test product. The appearance of relevant skin conditions (for example, redness) are analyzed using color data processing (CIE L*a*b* Color Scale) and/or geometrical quantification of areas corresponding to the respective condition in relative units (px). A Student's paired t-test analysis is conducted (where applicable) in order to compare the results of post-test treatment to baseline measurements. Statistical significance is established if p≤(α=0.05) is calculated.

Deliverable Final Report and Photographs

At the conclusion of the test period a final report is compiled consisting of a description of protocol and procedures described herein along with results of the visual evaluations, copies of the photographs on digital media, and tabular output generated from the qualitative questionnaire. Data will be treated at a 95% CL comparing baseline and post treatment for reduction in redness.

It is understood that the foregoing detailed description and accompanying examples are merely illustrative and are not to be taken as limitations upon the scope of the invention, which is defined solely by the following claims.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof

What is claimed is:

1. A skin care composition comprising:
(a) a colorant in an amount of about 0.01% to about 15.00% of the skin care composition by weight;
(b) an active agent in an amount of about 5.00% to about 20.00% of the skin care composition by weight;
(c) a film former in an amount of about 1.00% to about 30.00% of the skin care composition by weight; and
(d) an ointment base in an amount of about 15.00% to about 55.00% of the skin care composition by weight.

2. The skin care composition of claim 1, wherein the composition is essentially free of water.

3. The skin care composition of claim 1, wherein the colorant is selected from the group consisting of titanium dioxide, iron oxide, a clay mineral, carmine, a cosmetic dye, a pearlizing agent, and a combination thereof.

4. The skin care composition of claim 1, wherein active agent is an analgesic agent.

5. The skin care composition of claim 4, wherein the analgesic agent is selected from the group consisting of benzocaine, lidocaine, tetracaine, dibucaine, prilocaine, capsaicin, fentanyl, pregabalin, gabapentin, carbamazepine, oxcarbazepine, duloxetine, venlafaxine, bupropion, an opioid, and a combination thereof.

6. The skin care composition of claim 4, wherein the analgesic agent is benzocaine.

7. The skin care composition of claim 7, wherein the active agent is in an amount of about 10.00% of the skin care composition by weight.

8. The skin care composition of claim 1, wherein the film former is selected from the group consisting of 4,4′-diisocyanatodicyclohexylmethane polypropylene glycol polymer, behenyl olivate, behenyl erucate, dibehenyl fumerate, bis-behenyl/isostearyl/phyosteryl dimer dilinoleyl dimer dilinoleate, phytosteryl/behenyl/octyldodecyl lauroyl glutamate, bis-behenyl/phytosteryl dimer dilinoleate, and a combination thereof.

9. The skin care composition of claim 1, wherein the film former is a combination of polyolprepolymer-14 and behenyl olivate.

10. The skin care composition of claim 10, wherein the film former is in an amount of about 8.00% of the skin care composition by weight.

11. The skin care composition of claim 1, wherein the ointment base is petrolatum.

12. The skin care composition of claim 1, wherein the ointment base is in an amount of about 45.00% of the skin care composition by weight.

13. The skin care composition of claim 1, further comprising an additive selective from the group consisting of a surfactant, an oil absorber, a slip modifier, a flavorant, and a combination thereof.

14. The skin care composition of claim 13, wherein the skin care composition further comprises a surfactant selected from the group consisting of PEG-300, PEG-600, PEG-800, PEG-1000, PEG-2000, PEG-5000, PEG-10000, and a combination thereof.

15. The skin care composition of claim 14, wherein the surfactant is in an amount of about 5.00% to about 25.00% of the skin care composition by weight.

16. The skin care composition of claim 13, wherein herein the skin care composition further comprises an oil absorber, which comprises calcium silicate.

17. The skin care composition of claim 16, wherein the oil absorber is in an amount of about 1.50% to about 15.00% of the skin care composition by weight.

18. The skin care composition of claim 13, wherein the skin care composition further comprises a slip modifier, which is a powder comprising mica and titanium dioxide, a polymer microsphere comprising hexamethylene diisocyanate (HDI)/trimethylol hexyllactone crosspolymer and silica, calcium sodium borosilicate, polymethyl methacrylate, or a combination thereof.

19. The skin care composition of claim 18, wherein the slip modifier is in an amount of about 2.00% to about 30.00% of the skin care composition by weight.

20. The skin care composition of claim 18, wherein the slip modifier is a mixture of
a powder comprising mica and titanium dioxide, in an amount of about 1.00% to about 15.00% of the skin care composition by weight; and
a polymer microsphere comprising hexamethylene diisocyanate (HDI)/trimethylol hexyllactone crosspolymer and silica, in an amount of about 1.00% to about 15.00% of the skin care composition by weight.

21. The skin care composition of claim 1, comprising
(a) a colorant in an amount of about 1.00% to about 10.00% of the skin care composition by weight;
(b) benzocaine in an amount of about 7.00% to about 20.00% of the skin care composition by weight;
(c) polyolprepolymer-14 and behenyl olivate, each independently in an amount of about 1.00% to about 10.00% of the skin care composition by weight; and
(d) petrolatum in an amount of about 40.00% to about 50.00% of the skin care composition by weight.

22. A method of treating a skin condition comprising applying an effective amount of the skin care composition of claim 1 to a skin of a subject in need thereof.

23. The method of claim 22, wherein the subject has cold sore.

24. The method of claim 22, wherein the active agent of the skin care composition is benzocaine.

25. A method of treating cold sore comprising applying an effective amount of a skin care composition of claim 21 to a skin of a subject in need thereof.
